(19) United States
(12) Patent Application Publication
(10) Pub. No.: US 2024/0033501 A1
(43) Pub. Date: Feb. 1, 2024

(54) GARMENTS FOR ELECTRICAL STIMULATION

(71) Applicant: BIO-MEDICAL RESEARCH LIMITED, Galway (IE)

(72) Inventors: Conor MINOGUE, Galway (IE); Chandra LAKSHMANAN, Galway (IE); Maggie MCBRIDE, Galway (IS); Fiona O'Sullivan, Galway (IE)

(73) Assignee: BIO-MEDICAL RESEARCH LIMITED, Galway (IE)

(21) Appl. No.: 18/021,358

(22) PCT Filed: Aug. 9, 2021
(86) PCT No.: PCT/EP2021/072187

§ 371 Date: Feb. 14, 2023

(30) Foreign Application Priority Data
Aug. 14, 2020 (GB) 2012763.5

(51) Int. Cl. A61N 1/04

(52) U.S. Cl. CPC A61N 1/0484; A61N 1/0452; A61N 1/0456

(57) ABSTRACT

The present invention provides a compression garment for transcutaneous or neuromuscular electrical stimulation. The garment is adapted to be in contact with a part of the body. The garment is made from an elastic stretchable material. The garment comprises at least one compression band having a first portion and a second portion. The second portion is adapted to be coupled with an electrode, a portion of an electrode or a plurality of electrodes for contacting skin in use. The second portion has lower elasticity compared to that of the garment material of the first portion or no elasticity. Modulus of elasticity of the garment material in elongation direction and/or resting length of the garment material in the elongation direction in the first portion are selected so that pressure exerted by the second portion on a wearer when the garment is in use is equal or greater than a minimum predetermined pressure.

### FIELD OF THE INVENTION

The present invention relates to garments that incorporate electrical elements.

### BACKGROUND TO THE INVENTION

Transcutaneous electrical stimulation (TES) and neuromuscular electrical stimulation (NMES) are long established and widely used therapies that involve passing low level electric currents through skin contacting electrodes to activate underlying nerves. The electrodes typically have an area of skin contact ranging from 3 to 200 cm², with electrodes in the range 25 to 100 cm² being more popular. The electrodes serve to disperse the current at the skin so that the current density is reduced, thereby avoiding hotspots and reducing the likelihood of activating neural pain receptors in the skin. It is important that each electrode remains in contact with the skin over its available contact area, especially when using a constant current control system, since otherwise the current would be concentrated into the reduced area of contact leading to increased current density. Coupling of the electric current into the skin requires an aqueous electrolyte and this can be provided as a liquid, spray or gel.

Tight fitting garments incorporating electrodes on the skin-facing surface for transcutaneous stimulation provide a convenient way to locate a plurality of electrodes on the body with repeatable positioning. Flexible electrodes on the skin-facing surface of a garment can be electrically coupled to moistened skin if there is appropriate pressure urging the electrode towards the skin. Various body suits and garments have become available which use various means to achieve electrode contact, including tighten-able straps, zips and other fasteners. These are inconvenient and costly so there is a need for a more intuitive garment which locates electrodes and provides the right contact pressure to achieve comfortable stimulation.

In such garments for electrical stimulation it is necessary to make a conductive connection between each electrode and a terminal of an electronic signal generator. In a tightly fitting garment it is preferable that these conductors be integrated into the garment so that they are protected from damage and also that the user cannot alter them. Ideally, the user would not even be aware of them and so it is desirable that they be flexible, stretchable and have a very low profile. Such electrical conductors can take the form of wires, conductive threads, printed conductive tracks, conductive textile strips, amongst others. These conductors are typically insulated to prevent current leakage. The electronic controller and its battery power source can be integrated into the garment as well, or alternatively is detachable from the garment to allow the garment to be washed. The attachment mechanism can comprise latches, clips, an interference fit or magnetic attachment.

Electrodes must be in good uniform contact with the skin to ensure adequate stimulation and to prevent electric current from concentrating in small areas as this can cause discomfort.

Circular knit technology is frequently used to produce compression garments and the elastic properties of the knitted fabric can be varied in different parts of the garment by changing the knit parameters including the stitch type, yarn weight, proportion of inlaid elastic yarn. For example, EP1959880 B1, Soerensen et al, provides such detail for an abdominal compression garment but does not deal with integrating skin contacting electrodes at specific locations for making electrical contact with the skin, together with wiring and connections and how all these affect compression. Circular knit compression garments can be seamless providing enhanced comfort to the wearer. US2018036531 A1, Schwartz et al, describes incorporating electrodes into garments and providing zones of partial or different compression, but does not discuss how good uniform contact of electrodes with the skin can be achieved.

In view of the above, it is an object of the present invention to alleviate and mitigate the above disadvantages.

### SUMMARY OF THE INVENTION

In a first aspect, the present invention provides a compression garment for transcutaneous or neuromuscular electrical stimulation, wherein
the garment is adapted to be in contact with a part of the body;
the garment is made from an elastic stretchable material;
the garment comprises at least one compression band having a first portion and a second portion;
wherein the second portion is adapted to be coupled with an electrode, a portion of an electrode or a plurality of electrodes for contacting skin in use;
wherein the second portion has lower elasticity compared to that of the garment material of the first portion or no elasticity; and
wherein modulus of elasticity of the garment material in elongation direction and/or resting length of the garment material in the elongation direction in the first portion are selected so that pressure exerted by the second portion on a wearer when the garment is in use is equal or greater than a minimum predetermined pressure.

In an embodiment, the garment comprises at least two compression bands;
wherein each of the at least two compression bands has a first portion and a second portion;
wherein the second portion of each compression band is adapted to be coupled with an electrode, a portion of an electrode or a plurality of electrodes for contacting skin in use;
wherein the second portion has lower elasticity compared to that of the garment material of the first portion or no elasticity, and elasticity and/or size of the second portion of one compression band is different from the elasticity and/or size of the second portion of the other compression band; and
wherein in each of said at least two compression bands modulus of elasticity of the garment material in elongation direction and/or resting length of the garment material in the elongation direction in the first portion are selected so that pressure exerted by the second portion on a wearer when the garment is in use is equal or greater than a minimum predetermined pressure in each of said at least two compression bands.

Preferably, in each of said at least two compression bands the modulus of elasticity of the garment material in elongation direction and/or the resting length of the garment material in the elongation direction in the first portion are selected so that pressure exerted by the second portion on a wearer when the garment is in use is substantially the same in each of said at least two compression bands.

Preferably, the second portion comprises an electrode, a portion of an electrode or a plurality of electrodes. Preferably, the or each electrode is for contacting skin in use. It is often the case that the electrodes in different compression bands of a compression garment have different shapes, different contact areas, vary in number and/or are positioned relative to one another such that the length of the second portion in the elongation direction varies between the different compression bands. It is also possible for a single electrode of a particular shape being positioned across two or more compression bands, so that the second portion in the elongation direction varies between the different compression bands.

Preferably, the compression band extends in the elongation direction. Typically, in a compression garment the elongation direction is the direction of stretch which contributes most to providing pressure on the body. For example, in a tubular garment, the elongation direction is the circumferential direction, whilst elongation in the axial direction contributes little to providing tension or pressure. Preferably the at least two compression bands are juxtaposed in axial direction, i.e. in a direction transverse to the elongation direction. The garment may be tubular when in use. Preferably, the compression band extends in circumferential direction. Preferably, compression is mainly provided by the garment being stretched in circumferential direction when worn.

Preferably, the minimum predetermined pressure is at least 1 mmHg (1.33 hPa). The inventors have found that electrode contact pressure of at least equal or greater than 1 mmHg (1.33 hPa) provides excellent results allowing comfortable and effective electrical stimulation. An upper limit of 20 mmHg (26.66 hPa) is desirable to prevent discomfort. Accordingly, in the compression band, or as the case may be, in each of said at least two compression bands, the modulus of elasticity of the garment material in elongation direction and/or the resting length of the garment material in the elongation direction in the first portion are selected so that pressure exerted by the second portion on a wearer when the garment is in use is preferably in the range between 1 to 20 mmHg (1.33 hPa to 26.66 hPa). Preferably, the pressure is in the range between 1 to 20 mmHg (1.33 hPa to 26.66 hPa) when the garment material is stretched between 10% and 30% of resting length. Further preferably, the minimum determined pressure is at least 4mmHg (5.33 hPa) because this provides a greater margin to accommodate tissue compressability or differences in body shape.

Preferably, the garment material can elastically extend to 100% of resting length to facilitate donning and doffing.

The garment preferably includes wiring for the electrodes and a connector for connecting the wiring to a power source.

In one variation, the modulus of elasticity of the garment material in the elongation direction in the first portion is selected so that the resting length of the garment material in elongation direction in the first portion remains the same as in the first portion of the same length in a compression band where the elasticity of the second portion is the same as that of the first portion. Preferably, pressure exerted by the second portion on a wearer when the garment is in use is substantially the same in each of said at least two compression band. Preferably, the modulus of elasticity in the elongation direction in the first portion is further adjusted in accordance with the girth of a size model; the smaller the girth the higher the modulus.

In another variation, the resting length of the garment material in the elongation direction in the first portion is selected so that the modulus of elasticity in the first portion remains the same as in the first portion of the same length in a compression band where the elasticity of the second portion is the same as that of the first portion. Preferably, pressure exerted by the second portion on a wearer when the garment is in use is substantially the same in each of said at least two compression bands. Preferably, the resting length of the garment material in the elongation direction in the first portion is further adjusted in accordance with the girth of a size model; the smaller the girth the smaller the resting length.

The overall resting length of each compression band in elongation direction can differ from one another in order for the garment to conform to non-straight body shapes.

Elasticity in the context of the present invention should be generally understood as the ability of garment material to be stretched, i.e. elongated, upon application of an external force, and to return to the original un-stretched mode, i.e. to its resting length, upon withdrawal of the external force.

It is important that skin contact skin impedance does not exceed a predetermined value and/or the electrodes achieve a minimum contact pressure. This is achieved by producing sufficient garment tension. Garment tension is determined in part by the elongation from resting length and by the modulus of elasticity. These are the design variables which can be adjusted to achieve a uniform tension with variation in body size and restrictions in textile stretch. For a TES or an NM ES garment having a tubular section over its length in order to achieve uniform tension it is necessary to provide portions of unequal elasticity in different bands which encircle a body segment. Attachment of electrodes and wiring to the textile material, a typical material of compression garments, can significantly affect its mechanical stretch properties, sometimes even rendering it effectively non-stretch. In a portion of a garment containing electrodes, e.g. in a tubular portion, it is likely that a significant portion of the circumferential length of a band containing electrodes has relatively low stretchability due to the presence of the electrodes. Even flexible electrodes generally have a much higher modulus than the fabric onto which they are applied, and therefore greatly limit the stretchability of the fabric. Some printed electrodes do provide moderate degree of stretch but even still it is much more limited than the stretchability of the fabric. The required tension at the electrode over the intended size range of the garment is achieved in the present invention by adjusting the modulus of elasticity and/or resting length of the garment material in the band containing electrodes.

The present invention further identifies the required electrode pressure range that is required and how this is achieved in the design of the garment for specific electrode locations. It provides examples of multi-electrode configurations on different body parts illustrating how the necessary contact pressure is provided for different anatomies. It also provides the means by which different sizes of user can be accommodated.

The provision of the predetermined pressure of the second portion on the user's skin when the garment is worn ensures good contact of the electrode with the skin. As a result, skin contact impedance of the electrode does not exceed a level which may cause discomfort. The provision of uniform pressure across a plurality of electrodes ensures that the skin contact impedances of each of the plurality of electrodes are substantially uniform. Uniform contact pressure, that is at or above a predetermined pressure, ensures that the contact impedance is below the required level, and substantially uniform across the area of the electrode. This ensures uniform current distribution throughout the electrode which is the main determinant of comfort and effectiveness. If the contact impedance is locally too high, the effective conductive area of the electrode is reduced and in a constant current stimulation system this leads to a high current density hotspot which is the source of discomfort. In a constant voltage system, the increased contact impedance causes a reduction in current and therefore effectiveness. In either case it is desirable to maintain low contact impedance that is uniform within and between electrodes.

In a variation, the compression band, or, as the case may be, at least two compression bands cover a user's trunk in use and the second portion or portions are located over one of the following: the abdomen; the lower back; the lower back and the abdomen. Preferably, the garment comprises at least four electrodes, one located in a first compression band in the upper centre of the abdominal area, one located in a second compression band at the lower centre of the abdominal area and one in a third compression band on each side of the abdominal area between the rib cage and the respective pelvic bone.

In another variation, the at least two compression bands cover a user's pelvic area in use and the second portions are located over the buttocks.

In another variation, the compression band, or, as the case may be, at least two compression bands cover part of a user's leg in use and the second portions are located over one of the following: the upper leg muscles; the lower leg muscles; the upper leg muscles and the lower leg muscles.

The or each electrode may have an oval or rounded trapezoidal, or rounded triangular shape having a wide end and a narrow end, where the wide end is directed towards the centre of the abdominal area in use and the narrow end points away.

The garment may be made by circular knitting and the modulus of elasticity and/or the resting length of each compression band is controlled by varying knit parameters.

A receptacle may be formed on the outside of the garment for detachably attaching the power source.

The garment may comprise one or more compression bands only having a first portion and not having a second portion. Preferably, in a compression band not having the second portion, modulus of elasticity of the garment material in elongation direction and/or resting length of the garment material in the elongation direction are selected so that pressure exerted by the garment on a wearer when the garment is in use is smaller than the pressure exerted by the second portion of a compression band having the second portion.

In a second aspect, the present invention provides a kit of parts comprising:
a compression garment; and
one or more electrodes,
   as described above.

Features of the first aspect of the invention can be incorporated into the second aspect of the invention as appropriate and vice versa.

References to an “electrode” should be understood as including references to a portion of an electrode or a plurality of electrodes, unless otherwise specified.

### DETAILED DESCRIPTION OF THE INVENTION

The invention will now be described with reference to the accompanying drawings, which show, by way of example only, embodiments of the invention. In the drawings:

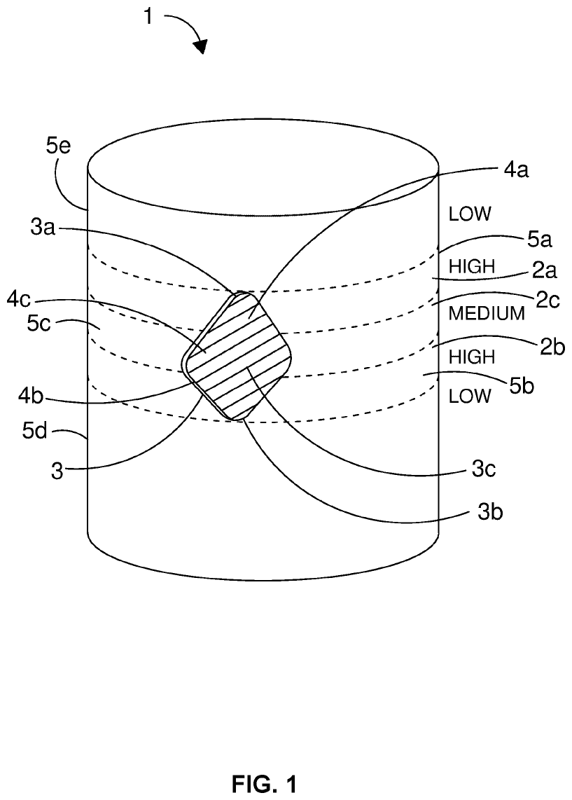

FIGS. 1 to 10 are schematic views of garments in accordance with the invention showing various compression bands and electrode configurations.

Compression garments are well known and work simply on the basis that the resting circumferential length of a stretchable tube portion of the garment is less than the girth of the body segment to which it is applied. To achieve compression, it is the stretch in the circumferential direction that counts, while stretch in the longitudinal direction contributes little if there is no curvature in that direction. When worn the garment is therefore stretched elastically and the resultant tangential tension results in an inward force, creating pressure. The amount of pressure at a given point depends on the modulus of elasticity of the fabric, the circumferential elongation or strain and the curvature of the fabric at the given point.

In an elasticated fabric tube, having constant linear modulus of elasticity in the circumferential direction throughout, that is fitted onto a cylindrical shaped body part, the pressure P at a point on the body depends on the tension of the fabric and the radius of curvature of the surface at that point.

P=T/ρ Equation 1

where T is the tension per unit width of the fabric (N/m) and ρ is the radius (m) of the cylinder at that point. The tension that is developed in the textile depends on its elongation and its modulus of elasticity.

T=E(C₁−C₀)/C₀ Equation 2

where C₁ is the circumference of the garment when stretched on the body and C₀ is its resting circumference. The ratio (C₁−C₀)/C₀ is a measure of the elongation E of the material. E is the modulus of elasticity of the textile, in this case expressed in units N/m. The modulus of a textile sample is measured by noting the force required to stretch a sample of known width. For example, a force of 20N is required to stretch a sample (length 30cm×width 10 cm) length-wise by 15 cm between two grips that are 10 cm wide. By rearranging Equation 2, the modulus Eat that level of elongation is therefore (20/0.1)/(0.15/0.3)=400N/m.

The inventors have found that pressure in the range 1 to 20 mmHg (1.33 hPa to 26.66 hPa) at the electrodes is sufficient to achieve the necessary contact electrical impedance. In practice, it is preferred to target a pressure of greater than 4mm Hg (5.33 hPa) to accommodate skin compressibility and shape variations. The pressure should be in this range for the entire skin contact area of each electrode, therefore the tension in the fabric supporting the electrodes needs to be sufficient. The tension achieved depends on the modulus of the fabric and the elongation when worn. This tension should be achieved when a given size garment is worn by all persons within the range for that size. Multiple garments having different sizes are required to accommodate the wider population.

Extension is simply, (C₁−C₀)/C₀ . measured in metres. The elongation ϵ relates the extension to the relaxed length ϵ=(C₁−C₀)/C₀. The garment industry tends to use the term “reduction factor” which expresses the amount of stretch as a percentage of the stretched length, i.e. F=100×(C₁−C₀)/C₁.

As an example, consider a compression garment intended to give pressure within a defined range (1.33 hPa to 26.66 hPa) at the abdomen. Assume the intended size range is from 76 to 81 cm girth at the waist. To achieve the pressure of 4 mmHg or 5.33 hPa (533 N/m²) at the lower end of this range, according to Equation 1, the tension required is 533×0.2=64 N/m. This assumes a radius of 0.12 m, corresponding to a cylindrical girth of 76 cm. Therefore, it is required that an elongation to 76 cm gives at least the required minimum tension. Assuming a uniform modulus of 500 N/m throughout the circumference, an elongation ϵ of about 0.13 is required to generate this tension, Equation 2, leading to a resting girth of 67 cm. The garment must be capable of elastically stretching to the upper limit of the size range, 81 cm, an elongation of 21%, without exceeding the upper tension limit. To allow the garment to be put on and taken off, it needs to be able to stretch over the shoulder or hip area without requiring abnormal strength on the part of the user. This could be an elongation of 100% and the fabrics needs to recover quickly when relaxed. Of course, the cross section of the body part may not be circular and an elliptical form may provide a more appropriate model, as detailed in EP 1959880 B1, Soerensen et al, for the abdomen.

A particular complication arises due to attachment of electrodes and wiring to the textile material which can significantly affect its mechanical stretch properties, sometimes even rendering it effectively low-stretch or non-stretch. If we consider a tubular portion of a garment containing electrodes it is likely that a significant portion of the circumferential length has relatively low stretch due to the presence of the electrodes. Flexible electrodes generally have a much higher modulus than the fabric onto which they are applied, and greatly limit the stretch. Some printed electrodes do provide moderate degree of stretch but even still it is much more limited than the stretch fabric. Therefore, achieving the required tension at the electrode over the intended size range means that the modulus and/or extension of the textile needs to be adjusted.

The attachment of electrodes and wiring onto or within the textile creates distortions in the garment when worn. There are shear areas at the edge of the attachment region where there are large changes in elasticity. There is a tendency for the edge of the electrode to lift from the skin near such transitions. The addition of low-stretch or non-stretch regions means the remaining stretched regions are subjected to higher elongation relative to resting length and therefore result in higher tension. This extra tension can create problems for the user and potentially cause garment damage.

### Adjustments to Compression Garment Properties to Accommodate Electrodes

There are two principal ways in which the garment design can be adjusted in accordance with the invention to achieve the target pressure while compensating for the addition of high modulus or non-stretch electrodes and wiring material.

First alternative: Keep modulus of textile used the same, change the reduction factor. If m is the length of the non-stretch material, then, C₀-m is the resting length of the stretch material. When the overall assembly is stretched to a length of C₁ then the elongation of the stretch material is

(C₀−m)/(C₀−m)=1+ϵ

Therefore, to achieve the same tension with the same modulus, when stretched to a known girth C₁, the relaxed girth has to be

C₀=(C₁+ϵm)/(1−ϵ) Equation 3

In the example above, assume that 35 cm of the resting girth is taken up of electrodes that have little or no stretch. Therefore, the elongation and tension must be provided by a fabric length of 76−35=41 cm. Assuming the same modulus as previously the resting girth of the garment is calculated by Equation 3 as 71.3 cm.

Second alternative. Keep elongation of the material the same, change modulus of textile. In this case the resting and extended length is fixed and the modulus of the stretch material has to be reduced to account for the length m of non-stretch material.

E′=(C₁−m)E/C₁ Equation 4

where E is the original modulus when m=0, and E′ is the adjusted modulus when m>0. In other words, the modulus is reduced when m>0.

The above principle relates to a situation where part of the circumferential path is non-stretch due to the presence of electrodes and associated wiring. For the situation where these structures are stretchable to some extent then the equations above have to be adjusted to allow for part of the extension when stretched to come from a first portion that contains electrodes or wiring and the remaining extension from a second portion that does not contain electrodes or wiring. Since the portions are in series circumferentially the tension is the same in both portions then

T=Eₐϵₐ=E_{b}ϵ_{b} Equation 5

where Eₐ,ϵₐ are the modulus and elongation of a first portion a and E_{b},ϵ_{b} are the modulus and elongation of a second portion b. Therefore,

Eₐ/E_{b}=ϵ_{b}/ϵₐ Equation 6

It is possible therefore to calculate the extension of each portion. It is also likely that the electrodes and wiring have a non-linear extension-tension relationship and so the contribution to stretch would vary with overall elongation. In any case the properties of the base garment have to be adjusted to compensate for the addition of electrodes while achieving the target electrode pressure.

The tension in the garment when worn is determined by the modulus of the fabric and the elongation of the fabric.

It is often the case that the electrodes within a circumferential band of a compression garment have shapes and/or are positioned relative to one another such that the circumferential length of non-stretch material varies across the width of the band. See garment 1 in FIG. 1 where typically the value m at the upper and lower ends 3a, 3b of an electrode 3 is much less than at the centre. There is more elasticated material in upper and lower compression bands 5a, 5b and their relative elongation is less than that of the elastic material of a compression band 5c at the centre, leading to reduced tension and therefore reduced electrode pressure. The electrode 3 therefore tends to lift from the skin at the ends 3a, 3b. In accordance with one alternative of the invention, to compensate, the resting girth in the compression bands 5a, 5b can be adjusted in line with Equation 3 above for the case where the modulus in the compression bands 5a, 5b is kept constant. In accordance with another alternative of the invention, the resting circumference of the compression bands 5a, 5b can be retained and the modulus can be increased in compression bands 5a, 5b according to Equation 4. FIG. 1 shows how the modulus is high in respective first portions 2a, 2b of the compression bands 5a, 5b containing the electrode ends 3a, 3b in respective second portions 4a, 4b but is medium in the first portion 2c of the compression band 5c at the centre containing a central part 3c of the electrode 3c in the second portion 4c. The modulus is preferably lower in compression bands 5d, 5e not containing the electrode 3.

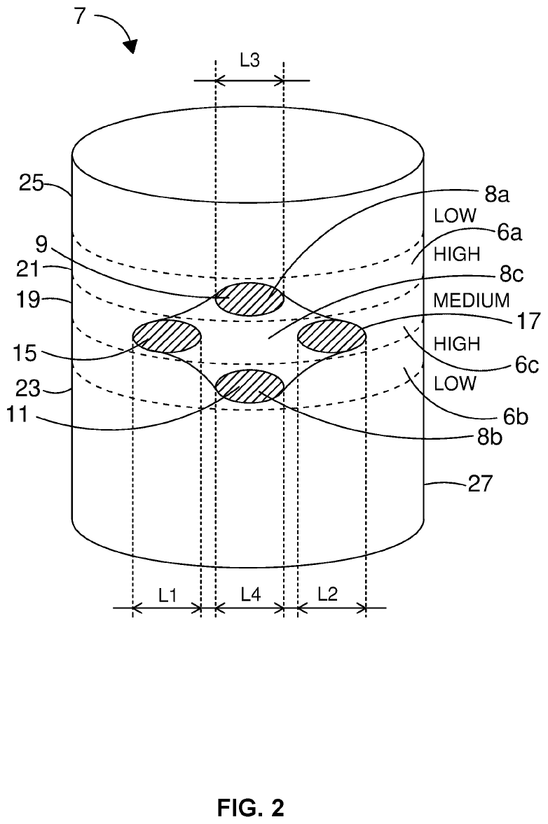

FIG. 2 illustrates a tubular section of a garment 7 with four abdominal electrodes 9, 11, 15, 17 in second portions 8a, 8b, 8c of respective bands 21, 23, 19 in a diamond pattern such that the length 11+12 of a non-stretch portion in the central compression band 19 is much higher than the non-stretch length 13 and 14 at in the upper and lower compression bands 21, 23. In accordance with the invention, the modulus of the fabric in the first portion 6c of the central compression band 19 is reduced relative to that in the first portion 6a, 6b of the upper and lower compression bands 21, 23 so that the pressure at the portions of the compression bands 19, 21, 23 containing the electrodes 9, 11, 15, 17 remains uniform and above the target level. The modulus of the fabric in compression bands 25, 27 not containing the electrodes 9, 11, 15, 17 is lower than in the first portion 6a, 6b, 6c of any compression band 19, 21, 23.

The same principle applies to any electrode whose length in elongation direction varies in the direction transverse to the elongation direction in an elasticated garment or a pattern of electrodes where the number of electrodes is different in different compression bands.

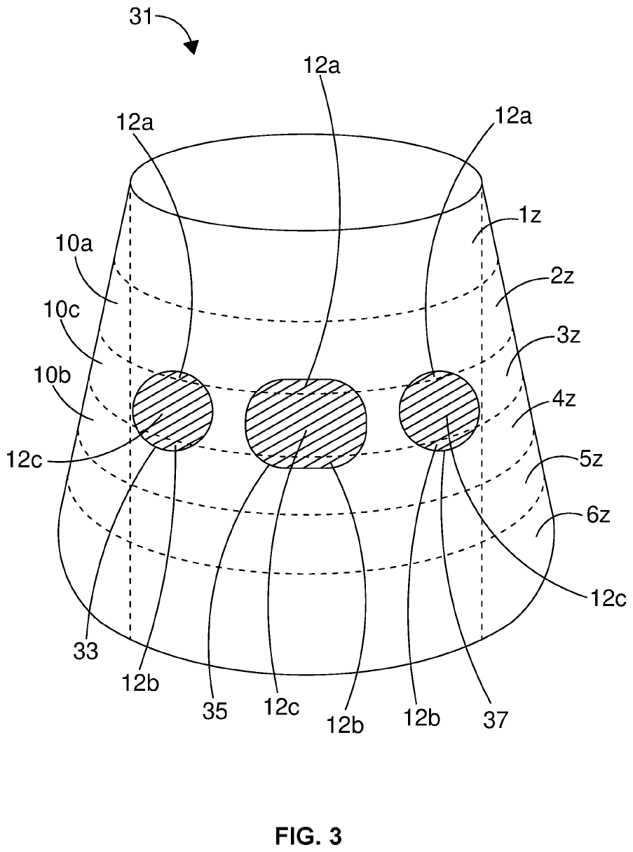

A similar situation arises when the body girth changes within an area of the garment containing electrodes. For example, the female form is characterised by a smaller girth at the waist which increases towards the hip area. The fabric is therefore stretched less at the waist leading to a lower tension. Accordingly, the modulus at the waist may be increased to achieve a higher tension, or alternatively the resting girth of the garment may be reduced at the waist such that it is stretched more relative to its flat or resting length. In general, where a body segment is non-cylindrical and a constant pressure is required from a substantially cylindrical elastic tube, then the modulus in the circumferential direction corresponding to the higher girth part of the body segment is less than that corresponding to a narrower part of the body segment. The modulus can be graded from the narrow to the wide girth parts. See FIG. 3 where a garment 31 comprises six compression bands 1Z-6Z illustrated in their stretched position. The modulus at compression band 1Z at waist region and not containing an electrode can be low, whereas the moduli of respective of first portions 10a, 10c, 10b compression bands 2Z, 3Z and 4Z containing portions of electrodes 33, 35, 37 in respective second portions 12a, 12b, 12c should be high, medium and medium-low respectively, leaving compression bands 5Z and 6Z to have low and very low modulus. An equivalent effect could be achieved by using a non-cylindrical garment where the resting length in each compression band 1Z-6Z is correspondingly adjusted.

One of the problems the invention addresses is uneven tension provided by a compression garment with built in electrodes and conductors by providing compression bands of unequal modulus and/or unequal resting length to account for the geometry and/or juxtaposition of the electrodes and the expected changes in girth of the subject.

As discussed above, the textile related factors contributing to garment tension and thereby pressure are modulus of elasticity and elongation. Although the pressure can be increased by either increasing the modulus or elongation it is preferable to limit the elongation when worn to approximately 30%. There are several reasons for this. For example, when a textile is stretched it becomes less opaque and therefore becomes partially “see through” when worn at high elongation. A stretched band of textile also becomes narrower causing distortion in the shape of the garment when worn. For this reason, for a garment incorporating electrodes for TES or NMES elongation of the textile should preferably be limited to about 30% when worn, even though higher levels of elongation are required for donning and doffing of the garment.

Body tissue is compressible to a certain extent and therefore the girth of a body segment with the applied pressure of a compression garment can be less than that without the garment. The resulting reduction in elongation of the fabric reduces the fabric tension and thereby the applied pressure. Thus, there is an equilibrium established between the compression of the tissue and the available pressure from the garment. In general, to compensate for this effect, the tension generated by the garment needs to be increased by a factor which takes account of the compressibility of the tissue. In the abdomen, for example, between the rib cage and the pelvis, the tissue is quite compressible. For pressures in the range 5 to 10 mmHg (6.7 to 13.33 hPa) the required increase in tension for a compressible form can be as high as double that required for a non-compressible form. Therefore, the modulus and/or reduction factor needs to be increased to take account of this effect. Over the bony region of the pelvis or ribs the tissue is less compressible, further underlining the need to have compression bands having different stretch properties.

In summary, for TES or NMES it is important that electrodes achieve a minimum contact pressure and this is achieved by producing sufficient garment tension in the compression bands. Garment tension is determined in part by the elongation from resting length and the modulus of elasticity. These are the design variables which can be adjusted to achieve a uniform tension with variation in body size and restrictions in textile stretch. For a TES or an NM ES garment having a tubular section over its length it is necessary to provide portions of unequal elasticity in different bands which encircle a body segment.

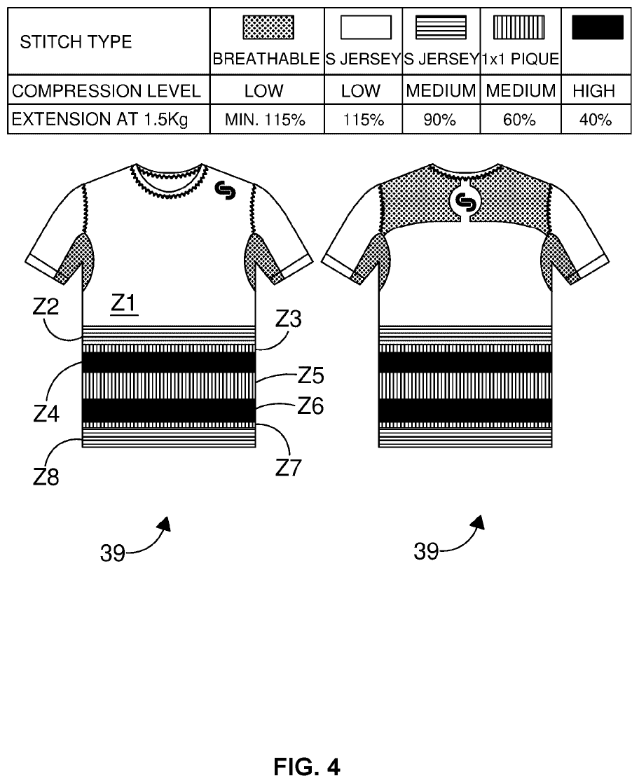

FIG. 4 shows a TES or NMES shirt 39 (electrodes not shown) that has a body section having several compression bands labelled Z1 through Z8, each compression band having a defined modulus of elasticity in the circumferential direction which may be different from other compression bands. See Table 1 for a summary of the material properties in this example and the key in FIG. 4 identifies the modulus used in each compression band. Each compression band Z1-Z8 may also have a resting circumferential length and width which is different from other compression bands.

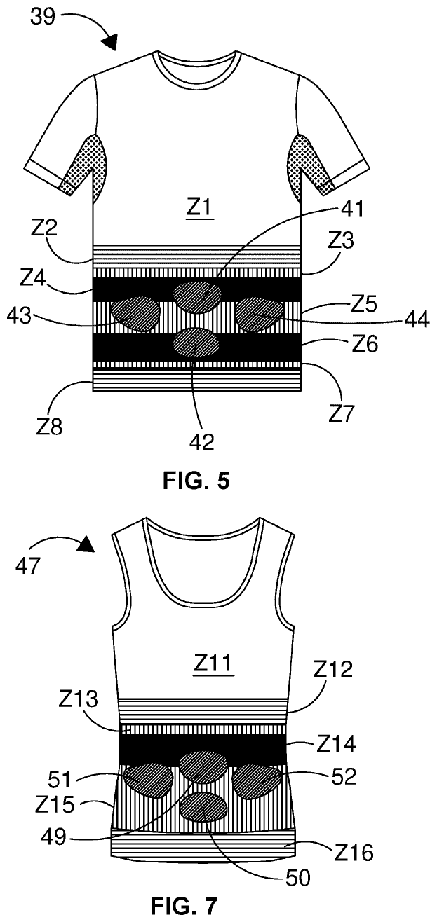

FIG. 5 shows the same male garment 39 having four stimulation electrodes 41, 42, 43, 44 positioned within the medium and high modulus compression bands Z4-Z6. The superior and inferior electrodes 41, 42 are mostly located in high modulus compression bands Z4, Z6 while the lateral electrodes 43, 44 are located mostly within the medium modulus compression band Z5. This arrangement ensures a consistent pressure on the electrodes 41, 42, 43, 44. The resting lengths of these compression bands are also slightly different and are arranged to give the correct range of tension for the size range covered by a given garment design. See Tables 4 and 5 for a summary of the size ranges and key dimensions of the compression bands for Male and Female garments.

Table 2 provides a simulation of the pressure created across the size range of a medium garment, assuming the girth range at the waist is from 83 to 92 cm.

There are also transition bands of intermediate modulus between the high compression bands and the low compression bands. This avoids abrupt transitions in elasticity while helping to decouple compression bands containing electrodes from the rest of the garment. This reduces the tugging on the electrodes which would otherwise occur due to movement of the arms and shoulders.

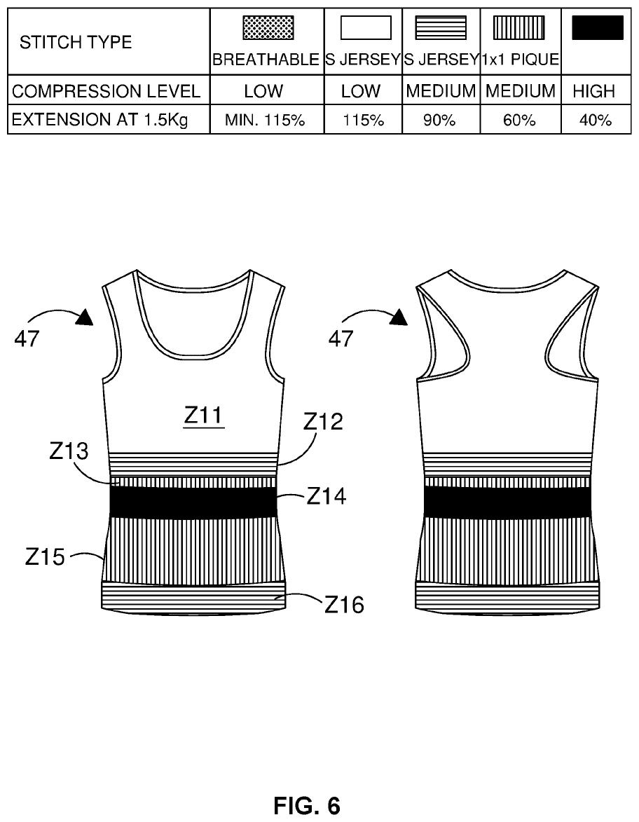

FIG. 6 shows a design for a female garment 47 (electrodes not shown). The arrangement of the compression bands Z11-Z16 is different compared to the male example in FIGS. 4 and 5 because the female hip girth is typically larger than the waist. This means the inferior high modulus compression band Z6 of the male garment of FIGS. 4 and 5 is not necessary because the material in this area is more elongated creating greater tension. FIG. 7 shows the placement of electrodes 49, 50, 51, 52 for the female garment 47.

It is possible to make a three-electrode design by combining both the superior and inferior electrodes (for example, electrodes 9 and 11 of FIG. 2) into one electrode 35 of FIG. 3 and relocating over the mid-point joining the centre of those electrodes, see FIG. 3. The area of the single electrode 35 should be typically 1.5 to 2 times the area of a side electrode 33, 37 to control the current density. A two electrode version could also be made by simply omitting the centre electrode 35 and just using the side electrodes 33, 37. This would simplify the compression bands that are required to support the electrodes but the effectiveness of the system to target specific parts of the muscle group would be reduced.

TABLE 1
Example material properties of textile
across different compression bands:

| | band | elongation @ | |
|---|---|---|---|
| Compression | code | 1.5 kg | E (N/m) |
| low | 1 | 1.15 | 213 |
| low to med | 2 | 0.90 | 273 |
| medium | 3 | 0.60 | 409 |
| High | 4 | 0.40 | 613 |

In this example, the garment was achieved by circular knitting and it was found useful to achieve compression bands 1 through 3 using a single jersey knit stitch and compression bands 4, 5 and 6 with a Pique stitch.

TABLE 2
Calculation of the pressure generated within different compression bands of the
garment at lower and upper limits of the medium size male garment. It assumes
circular cross section and that the electrodes are substantially non stretch:

| | | | LB | HB | | | | | | |
|---|---|---|---|---|---|---|---|---|---|---|
| | | | Low | High | ϵ of | ϵ of | | | | |
| | | flat | Bound | Bound | stretch | stretch | Tension | Tension | Pressure | Pressure |
| | E | width | Girth | Girth | part | part | @ LB | @HB | @ LB | HB |
| Band | code | (cm) | (cm) | (cm) | @LB | @HB | (N/m) | (N/m) | (mm Hg) | (mmHg) |
| 1 | 1 | 40.5 | 83.0 | 92.0 | 0.02 | 0.14 | 5.3 | 29.0 | 0.3 | 1.5 |
| 4 | 4 | 37.5 | 83.0 | 92.0 | 0.12 | 0.26 | 75.5 | 160.4 | 4.3 | 8.2 |
| 5 | 3 | 37.5 | 83.0 | 92.0 | 0.20 | 0.42 | 80.7 | 171.6 | 4.6 | 8.8 |
| 6 | 4 | 37.5 | 83.0 | 92.0 | 0.12 | 0.26 | 75.5 | 160.4 | 4.3 | 8.2 |
| 8 | 2 | 39.5 | 83 | 92 | 0.05 | 0.16 | 13.8 | 44.8 | 0.8 | 2.3 |

To cater for a wider population a range of sizes must be provided. Exemplary sizes and corresponding key dimensions for male and female population is provided in Tables 3 and 4 respectively.

TABLE 3
Exemplary male sizes and key dimensions. The compression band depth
refers to the combined width of compression bands 4, 5 and 6. The
“½ Chest” refers to the flat length of the
garment at the level of the chest in relaxed state, i.e. half of
its circumferential length. The cylinder refers to the size of the
circular knit cylinder that is used, where that is the technology used.

| Male | S | XL |
|---|---|---|
| CYLINDER (in) | 15.0 | 20.0 |
| ½ Chest (cm) | 35.5 | 48.0 |
| ½ Waist (cm) | 32.5 | 45.0 |
| band 5 | | |
| Compression | 21.5 | 23.5 |
| band depth cm | | |

TABLE 4
Exemplary female sizes and dimensions
of compression band. See FIG. 6.

| Female | XXS | L |
|---|---|---|
| CYLINDER (in) | 12.0 | 18.0 |
| ½ Chest (cm) | 28.0 | 43.0 |
| ½ Waist (cm) | 24.0 | 39.0 |
| Compression | 21.5 | 23.5 |
| band depth cm | | |

The elastic stretch property of the fabric is largely determined by the proportion of inlaid elastic yarn to the knitted fabric, for example, elastane. The ground yarn can be nylon or polyester. The thickness of the elastic yarn and the numbers per unit width can be adjusted to achieve the necessary elastic properties. Typically, the percentage of elastane is in the range 8 to 30% and preferably it is in the range 9 to 15%

The process of attaching wiring onto a garment may involve the application of considerable heat. It has been found that nylon 66 provides the necessary heat resistance to retain mechanical properties of the fabric post attachment. It is also preferable that the yarn be dope dyed rather than dying the completed garment. This is to eliminate dye residues that inevitably remain on the dyed garment, in particular on the electrodes.

Alternatively, a TES or an NMES garment can have a tubular section whose circumferential length varies to give varying degrees of elongation to compensate for variations in body segment girth. Such an approach may be more suited to “cut and sew” construction providing a more tailored garment.

Circular knit garments can provide variations in modulus of elasticity by controlling the knit density, the type of knitted stitch, yarn composition, yarn gauge, yarn tension. Garments can also be made by cutting sections of fabric having a range of mechanical properties and sewing them together to achieve regions of different modulus and range of elasticity.

The position of the electrodes on the internal, skin facing surface of the garment is critical to the delivery of electrical stimulation because when worn the electrodes are required to align with particular anatomical locations and to receive adequate compression. For abdominal stimulation, two, three or four electrodes locate on the anterior abdominal wall. The electrodes are typically positioned symmetrically with respect to the midline. The vertical position of the mid-point of the electrode array is typically centred at or near the umbilicus. These requirements translate to positions on a male garment as shown in FIG. 5 where the electrodes 41, 42, 43, 44 are located within respective compression bands Z4, Z5 and Z6. For a female garment, the electrodes 49, 50, 51, 52 are located within respective compression bands Z14, Z15, see FIG. 7. The location of the compression bands vertically (axially) varies depending on the gender and size of the garment. Tables 3 and 4 summarise the key dimensions for a range of sizes of a male and female garments respectively. The provision of the predetermined pressure of the electrodes on the user's skin when the garment is worn ensures good contact of the electrode with the skin. As a result, skin contact impedance of the electrode does not exceed a level which may cause discomfort. The provision of uniform pressure across a plurality of electrodes ensures that the skin contact impedances of each of the plurality of electrodes are substantially uniform. Skin contact impedance of the electrodes can be monitored by a suitable method to ascertain that it remains at an acceptable level. One such suitable method for monitoring skin contact impedance is described in the applicant's PCT/EP2019/058113 (WO2019/185934). Essentially, the method comprises forming an array comprising at least two electrodes; controlling flow of current pulses within electrode pairings of the array; measuring at least one voltage sample between electrodes at at least one time point within a stimulation pulse; calculating a time dependent factor based on the voltage sample or samples; and assessing the quality of electrode contact by: comparing the calculated time dependent factor with a pre-determined acceptance limit; characterising the quality of electrode contact as acceptable if the calculated time dependent factor is less than or equal to the pre-determined acceptance limit; and characterising the quality of electrode contact as unacceptable if the calculated time dependent factor is greater than the predetermined acceptance limit. A time dependent factor of voltage may be a calculation that takes as its input the magnitude of a sample or samples of a voltage across a pair of electrodes as well as the time within the pulse at which the sample or samples were taken. Examples could include the rate of change of voltage between two time points, the difference between each sample and an upper limit which itself varies with time, a time constant of the voltage waveform, a cross correlation function with a template waveform, a coefficient of a polynomial curve fit of the voltage waveform. The time dependent factor may also be adjusted by the magnitude of the current used in the pulse in order to calculate a further factor, for example to estimate the capacitance. Alternatively, the magnitude of the current can be used to adjust the acceptance limit. At least one voltage sample can be measured between electrodes at a plurality of time-points within the stimulation pulse. The time dependent factor can be one or more of the following: the difference between an initial voltage step and a voltage at a later time point in the stimulation pulse; the voltage at a defined later time point in the stimulation pulse; the difference between the initial voltage step and the voltage at the end of the pulse; an estimated time constant of a voltage waveform; an estimated rate of change of voltage with respect to time at a given time point; the electrode capacitance which is calculated by dividing an accumulated charge at a time point by a differential voltage at the time point; a coefficient of a polynomial model. The predetermined acceptance limit may be dependent upon the magnitude of a current selected for the stimulation pulse. The acceptance limit may be a maximum expected voltage value for the time point at a selected current within the stimulation pulse. The array may comprise at least three electrodes and a constant current could be driven between two of the at least three electrodes while sampling the voltage across these two electrodes and also at a third electrode, calculating the time dependent factor for the two electrodes and the third electrode, calculating a ratio of the time dependent factors and comparing the ratio with an acceptance limit. At least two electrode pairings of the array have a common electrode. The capacitance for each of the electrodes may be calculated and an electrode with the lowest capacitance can be identified as faulty. A plurality of voltages across each of the at least two electrode pairings of the array may be measured at a plurality of time points during the stimulation pulse. Voltages across each of three electrode pairings of the array may be measured at the plurality of time points during the stimulation pulse. At least one faulty electrode may be identified by comparing measured voltages across each of the at three electrode pairings with at least one reference value in order to identify a faulty electrode. At least one faulty electrode may be identified by calculating a voltage drop across at least one electrode and comparing the voltage drop to a predetermined acceptance limit.

When the garment is worn it is essential that the electrodes remain in their designated positions and not move when the person moves. For example, abdominal electrodes should remain in place with movement of the shoulders and arms, movement of the legs, flexion at the hip etc. It is necessary to decouple these movements from exerting forces on the electrodes. For example, the fabric under the arms is stretched when the arms are raised and therefore the modulus in this area should be low so that arm movement does not result in the abdominal part being pulled. It is therefore desirable that the garment has bands of varying compression and range of extension. In a preferred embodiment, a first tubular section of the garment containing one or more electrodes is decoupled from the rest of the garment by a second tubular portion having lower, intermediate, modulus of elasticity.

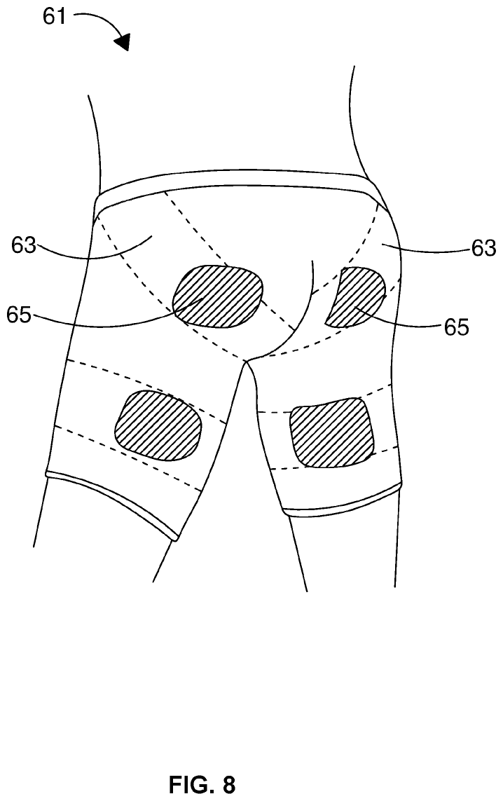

The gluteal area can have significant curvature in the vertical as well as the horizontal direction. Moreover, the cleft of the buttocks can result in significant changes in the magnitude and direction of the curvature, even within the horizontal plane (transverse) plane. Referring to FIG. 8, an electrode 65 placed on the buttock area may therefore require fabric tension at an angle to the horizontal. This can be provided, for example, by an elasticated band 63 that runs from a position on the posterior waistband of a pair of compression shorts 61 to the crotch area, this area being stabilised by continuing the elasticated band 63 to the waistband anteriorly, as illustrated in FIG. 8, or by restricting the stretch of the anterior part of the garment such that the crotch area is anchored and prevented from being pulled posteriorly.

The upper part of the legs can have an inverted conical shape and there can be a requirement to locate electrodes anteriorly over the quadriceps and/or posteriorly over the hamstrings. An elasticated portion between these electrodes, both medially and laterally, provides the necessary tension when stretched. For a cylindrical tubular garment section to achieve consistent pressure throughout its length in this case, the modulus of elasticity of the fabric in the horizontal direction for the narrower portion of the leg is greater than the upper part of the leg.

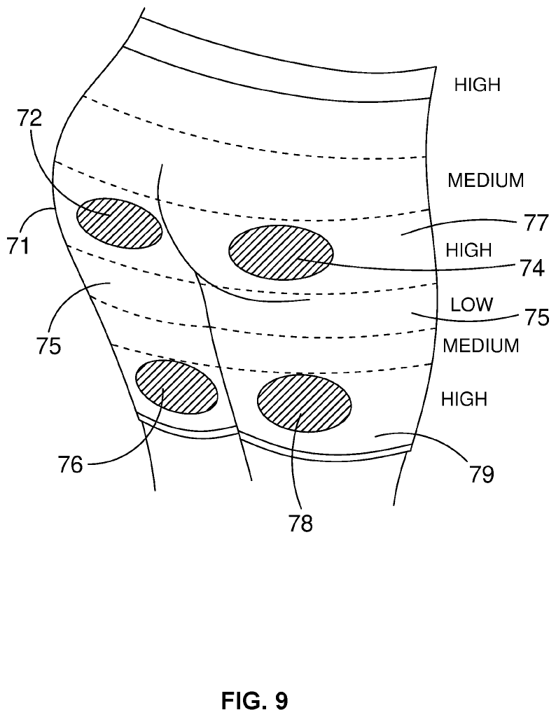

It is important to prevent movement of the legs from pulling on the fabric of the garment such as to cause movement of the electrodes relative to the skin. For this reason, the garment should stretch easily in the vertical direction around the hip joint. With reference to FIG. 9, this can be achieved by providing a horizontal band 75 in garment 71 that is effectively loose fitting, or where the modulus is very low, especially in the vertical direction, allowing the material to stretch in this region. A loose or low modulus band 75 at the top of each leg can provide the necessary decoupling between the legs and the pelvic area, so that compression bands 77, 79 on the legs and buttock areas containing electrodes 72, 74, 76, 78 are separated.

In general, where electrodes are located on body parts which move relative to one another around a joint it is necessary to prevent such movement exerting pulling forces on the electrodes. This also arises where a garment extends over a joint and where movement of the joint tends to pull on a section of the garment containing electrodes. Decoupling is achieved by providing the garment with low modulus stretch over the joint, especially in the direction of joint rotation. Any wires which traverse this area must also stretch or be routed in such a way as to enable stretch of the fabric, for example, by using a zigzag or wave pattern.

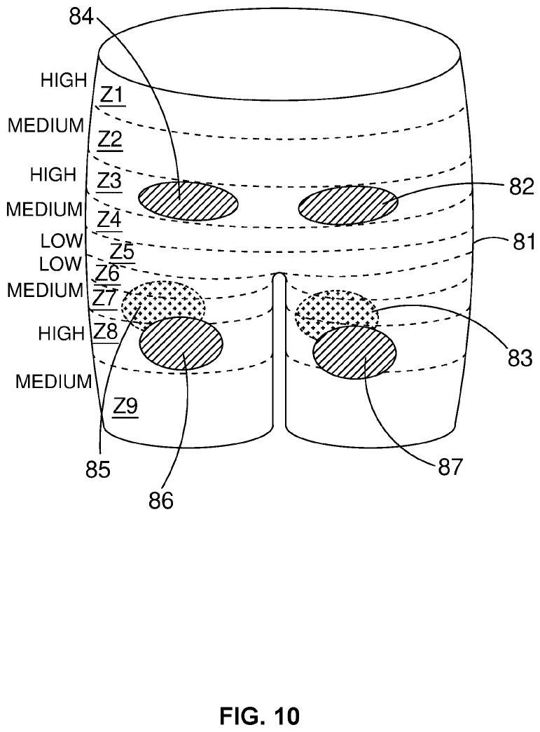

Another garment 81 with an arrangement of compression bands for stimulation of the buttocks and legs is shown in FIG. 10. Compression bands 25 and 26 provide decoupling between pelvic and leg areas by having low modulus of extension in the vertical direction. The waistband, compression band 21, is a high compression area, helping to anchor the whole garment 81. Compression bands 23 and 28 containing electrodes 82-87 provide high compression by having a higher modulus in the circumferential direction than the adjacent areas that do not contain electrodes.

It will be appreciated by those skilled in the art that variations and modifications can be made without departing from the scope of the invention as defined in the appended claims.

1. A compression garment for transcutaneous or neuromuscular electrical stimulation, comprising:
a garment,
wherein the garment is adapted to be in contact with a part of the body; the garment is made from an elastic stretchable material;
wherein the garment comprises at least one compression band having a first portion and a second portion;
wherein the second portion is adapted to be coupled with an electrode, a portion of an electrode or a plurality of electrodes for contacting skin in use;
wherein the second portion has lower elasticity compared to that of the garment material of the first portion or no elasticity; and
wherein modulus of elasticity of the garment material in elongation direction and/or resting length of the garment material in the elongation direction in the first portion are selected so that pressure exerted by the second portion on a wearer when the garment is in use is equal or greater than a minimum predetermined pressure.

2. A compression garment of claim 1, wherein the garment comprises at least two compression bands;
wherein each of the at least two compression bands has a first portion and a second portion;
wherein the second portion of each compression band is adapted to be coupled with an electrode, a portion of an electrode or a plurality of electrodes for contacting skin in use;
wherein the second portion has lower elasticity compared to that of the garment material of the first portion or no elasticity, and elasticity and/or size of the second portion of one compression band is different from the elasticity and/or size of the second portion of the other compression band; and
wherein in each of said at least two compression bands modulus of elasticity of the garment material in elongation direction and/or resting length of the garment material in the elongation direction in the first portion are selected so that pressure exerted by the second portion on a wearer when the garment is in use is equal or greater than a minimum predetermined pressure in each of said at least two compression bands.

3. A compression garment of claim 2, wherein in each of said at least two compression bands the modulus of elasticity of the garment material in elongation direction and/or the resting length of the garment material in the elongation direction in the first portion are selected so that pressure exerted by the second portion on a wearer when the garment is in use is substantially the same in each of said at least two compression bands.

4. A compression garment of claim 2, wherein the second portion comprises an electrode, a portion of an electrode or a plurality of electrodes.

5. A compression garment of claim 1, wherein the compression band extends in the elongation direction.

6. A compression garment of claim 1, wherein the compression band extends in a circumferential direction within the garment.

7. A compression garment of claim 1, wherein compression is mainly provided by the garment being stretched in circumferential direction when worn.

8. A compression garment of claim 2, wherein the at least two compression bands are juxtaposed in an axial direction that is transverse to the elongation direction.

9. A compression garment of claim 1, wherein the modulus of elasticity of the garment material in elongation direction and/or the resting length of the garment material in the elongation direction in the first portion are selected so that the minimum predetermined pressure exerted by the second portion on a wearer when the garment is in use is at least 1 mmHg (1.33 hPa) or greater.

10. A compression garment of claim 1, wherein the modulus of elasticity of the garment material in elongation direction and/or the resting length of the garment material in the elongation direction in the first portion are selected so that pressure exerted by the second portion on a wearer when the garment is in use is preferably in the range between 1 to 20 mmHg (1.33 hPa to 26.66 hPa).

11. A compression garment of claim 10, wherein the pressure is in the range between 1 to 20 mmHg (1.33 hPa to 26.66 hPa) when the garment material is stretched between 10% and 30% of resting length.

12. A compression garment of claim 1, wherein the modulus of elasticity of the garment material in elongation direction and/or the resting length of the garment material in the elongation direction in the first portion are selected so that the minimum predetermined pressure exerted by the second portion on a wearer when the garment is in use at least 4 mmHg (5.33 hPa).

13. A compression garment of claim 1, wherein the modulus of elasticity of the garment material in the elongation direction in the first portion is selected so that the resting length of the garment material in elongation direction in the first portion remains the same as in the first portion of the same length in a compression band where the elasticity of the second portion is the same as that of the first portion.

14. A compression garment of claims claims 1, wherein the resting length of the garment material in the elongation direction in the first portion is selected so that the modulus of elasticity in the first portion remains the same as in the first portion of the same length in a compression band where the elasticity of the second portion is the same as that of the first portion.

15. A compression garment of claim 1, wherein the compression band covers a user's trunk in use and the second portion is located over one of the following: the abdomen; the lower back; the lower back and the abdomen.

16. A compression garment of claim 1, wherein the compression band covers part of a user's leg in use and the second portions are located over one of the following: the upper leg muscles; the lower leg muscles; the upper leg muscles and the lower leg muscles.

17. A compression garment of claim 2, wherein the garment comprises at least four electrodes, one located in a first compression band in the upper centre of the abdominal area, one located in a second compression band at the lower centre of the abdominal area and one in a third compression band on each side of the abdominal area between the rib cage and the respective pelvic bone.

18. A compression garment of claim 2, wherein the at least two compression bands cover a user's pelvic area in use and the second portions are located over the buttocks.

19. A kit of parts comprising:
a compression garment, wherein the garment is adapted to be in contact with a part of the body; the garment is made from an elastic stretchable material;
wherein the garment comprises at least one compression band having a first portion and a second portion;
wherein the second portion is adapted to be coupled with an electrode, a portion of an electrode or a plurality of electrodes for contacting skin in use;
wherein the second portion has lower elasticity compared to that of the garment material of the first portion or no elasticity; and
wherein modulus of elasticity of the garment material in elongation direction and/or resting length of the garment material in the elongation direction in the first portion are selected so that pressure exerted by the second portion on a wearer when the garment is in use is equal or greater than a minimum predetermined pressure; and
one or more electrodes.
